(19) United States
(12) Patent Application Publication
(10) Pub. No.: US 2023/0383320 A1
(43) Pub. Date: Nov. 30, 2023

(54) METHOD FOR PRODUCING MONOUNSATURATED FATTY ACID HAVING 16 CARBON ATOMS

(71) Applicants: SHIZUOKA PREFECTURAL UNIVERSITY CORPORATION, Shizuoka-shi (JP); NATIONAL UNIVERSITY CORPORATION TOKAI NATIONAL HIGHER EDUCATION AND RESEARCH SYSTEM, Nagoya-shi (JP)

(72) Inventors: Hiroshi Kikukawa, Shizuoka-shi (JP); Toru Suzuki, Gifu-shi (JP)

(73) Assignees: SHIZUOKA PREFECTURAL UNIVERSITY CORPORATION, Shizuoka-shi (JP); NATIONAL UNIVERSITY CORPORATION TOKAI NATIONAL HIGHER EDUCATION AND RESEARCH SYSTEM, Nagoya-shi (JP)

(21) Appl. No.: 17/979,878

(22) Filed: Nov. 3, 2022

(30) Foreign Application Priority Data
May 27, 2022 (JP) 2022-086879

(51) Int. Cl. C12P 7/6409; A61K 8/36; A61Q 19/00; C12N 1/20; A23L 33/12

(52) U.S. Cl. CPC C12P 7/6409; A61K 8/361; A61Q 19/00; C12N 1/205; A23L 33/12; A61K 2800/10; A61K 2800/85; C12R 2001/01

(57) ABSTRACT

Provided is a method for producing a monounsaturated fatty acid having 16 carbon atoms by a method other than chemical synthesis. The method for producing a monounsaturated fatty acid having 16 carbon atoms comprises a step of culturing bacteria of the genus Bifidobacterium capable of producing a monounsaturated fatty acid having 16 carbon atoms in a liquid medium.

### BACKGROUND OF THE INVENTION

### Field of the Invention

The present invention relates to a method for producing a monounsaturated fatty acid having 16 carbon atoms using bifidobacteria.

### Description of the Related Art

Atopic dermatitis is a disease whose main lesion is pruritic eczema, with repeated exacerbations and remissions. It is known that most cases of atopic dermatitis develop in childhood and, once cured, recur with severe symptoms in adolescence. The number of patients with atopic dermatitis in Japan has been increasing every year, from about 350,000 in 2008 to about 510,000 in 2017 (2017, Ministry of Health, Labour and Welfare), and is developing into a societal issue.

In recent years, it has become clear that one of the factors contributing to atopic dermatitis is the induction of inflammation by Staphylococcus aureus. In addition to atopic dermatitis, this induction of inflammation by Staphylococcus aureus is also a factor in chronic dermatitis and skin rashes. Therefore, to treat, improve or prevent this type of dermatitis, the growth and activity of Staphylococcus aureus need to be suppressed. Antibiotics are usually used to suppress microorganisms such as Staphylococcus aureus, but because antibiotics have strong antibacterial activity, they also suppress beneficial microorganisms such as indigenous skin bacteria.

In the meantime, sapienic acid (6-cis-C16:1), a monounsaturated fatty acid having 16 carbon atoms, is known to have antibacterial activity against Staphylococcus aureus. It has been reported that this sapienic acid exhibits selective antibacterial activity, i.e., it exhibits strong antibacterial activity against Staphylococcus aureus, but not against Staphylococcus epidermidis, a closely related but beneficial bacterium contributing to the moisturization of the skin (Non Patent Literature 1: Toshihiro Nagao, Ayaka Uyama, Shigemitsu Tanaka, and Teizo Sugino, “Fatty Acid That Controls Skin Bacterial Flora”, Biotechnology, The Society for Biotechnology, Japan, October, 2020, Vol. 98, No. 10, pp. 525-528). Non Patent Literature 1 also reports that palmitoleic acid (9-cis-C16:1) and 7-cis-hexadecenoic acid (7-cis-C16:1), also monounsaturated fatty acids having 16 carbon atoms, exhibit selective antibacterial activity similar to sapienic acid. Therefore, it is expected that the use of monounsaturated fatty acids having 16 carbon atoms that exhibit selective antibacterial activity, i.e., exhibit antibacterial activity against harmful Staphylococcus aureus but not against beneficial Staphylococcus epidermidis, has the potential to treat, improve, or prevent dermatitis such as atopic dermatitis while maintaining healthy microflora in the skin.

### SUMMARY OF THE INVENTION

To use monounsaturated fatty acids having 16 carbon atoms for the treatment, improvement, or prevention of atopic dermatitis, and the like, application of external skin preparations such as emulsions and creams containing these types of fatty acids, or oral administration of food/beverage products containing these types of fatty acids are considered. Therefore, it is desirable to obtain monounsaturated fatty acids having 16 carbon atoms as natural ingredients or naturally derived ingredients that can be easily used in external skin preparations, food/beverage products. However, the natural resources from which these monounsaturated fatty acids having 16 carbon atoms are extracted are limited, and it is difficult to obtain monounsaturated fatty acids having 16 carbon atoms as natural ingredients or naturally derived ingredients. Monounsaturated fatty acids having 16 carbon atoms are thus generally synthetic materials produced by chemical synthesis and are difficult to use in external skin preparations, food/beverage products.

Therefore, the present invention was made in consideration of the above-mentioned points, the object of which is to provide a method for producing a monounsaturated fatty acid having 16 carbon atoms by a method other than chemical synthesis.

Inspired by the fermentative production by microorganisms, the present inventors have extensively searched for microorganisms capable of producing monounsaturated fatty acids having 16 carbon atoms and studied their efficient production through characterization. As a result, the present inventors have found that bacteria of the genus Bifidobacterium, so-called bifidobacteria, which are yogurt-producing bacteria and are commonly known as probiotics, produce monounsaturated fatty acids having 16 carbon atoms. The present invention has been completed based on these findings.

To solve the above problems, the method for producing monounsaturated fatty acids having 16 carbon atoms of the present invention comprises a step of culturing bacteria of the genus Bifidobacterium capable of producing monounsaturated fatty acids having 16 carbon atoms in a liquid medium. This allows production of monounsaturated fatty acids having 16 carbon atoms by a method other than chemical synthesis. Specifically, by culturing bacteria of the genus Bifidobacterium capable of producing monounsaturated fatty acids having 16 carbon atoms in a liquid medium, the fatty acid of interest accumulates in the cells of the bacteria of the genus Bifidobacterium that have efficiently grown in the liquid medium.

In addition, the bacterium of the genus Bifidobacterium in the production method of the present invention is preferably Bifidobacterium adolescentis, Bifidobacterium boum, Bifidobacterium sp. strain JCM7042 or a mutant strain thereof. This allows to select bacteria of the genus Bifidobacterium that are highly productive of monounsaturated fatty acids having 16 carbon atoms and are suitable for the production of the fatty acid of interest.

In addition, the mutant strain of the bacterium of the genus Bifidobacterium in the production method of the present invention is preferably the Bifidobacterium sp. strain AD2 (NITE BP-03576). This allows to select a novel bacterium of the genus Bifidobacterium, that is particularly highly productive of monounsaturated fatty acids having 16 carbon atoms and is suitable for the production of the fatty acid of interest.

In the method for producing monounsaturated fatty acids having 16 carbon atoms of the present invention, the monounsaturated fatty acid having 16 carbon atoms is preferably 7-cis-hexadecenoic acid. This allows to select a monounsaturated fatty acid having 16 carbon atoms that is produced by bacteria of the genus Bifidobacterium and exhibits selective antibacterial activity, i.e., exhibits antibacterial activity against harmful Staphylococcus aureus but not against beneficial Staphylococcus epidermidis.

In addition, the liquid medium in the production method of the present invention is preferably a liquid medium in which 0.001 to 1 g/L of L-cysteine or a salt thereof was further added to MRS medium. This allows to improve the productivity of monounsaturated fatty acids having 16 carbon atoms, particularly 7-cis-hexadecenoic acid, by bacteria of the genus Bifidobacterium.

In addition, the culture in the production method of the present invention is preferably performed by aerating the liquid medium with carbon dioxide. This allows to improve the productivity of monounsaturated fatty acids having 16 carbon atoms, particularly 7-cis-hexadecenoic acid, by bacteria of the genus Bifidobacterium, and furthermore, to efficiently produce monounsaturated fatty acids having 16 carbon atoms even under large scale culture conditions.

The culture in the production method of the present invention is preferably performed under temperature conditions of 32 to 35° C. This allows to improve the productivity of monounsaturated fatty acids having 16 carbon atoms, particularly 7-cis-hexadecenoic acid, by bacteria of the genus Bifidobacterium.

The method for producing a selective antibacterial agent for Staphylococcus aureus of the present invention comprises a step of culturing bacteria of the genus Bifidobacterium capable of producing a monounsaturated fatty acid having 16 carbon atoms, and the monounsaturated fatty acid having 16 carbon atoms is 7-cis-hexadecenoic acid. The bacteria of the genus Bifidobacterium produce 7-cis-hexadecenoic acid, which allows to obtain a selective antibacterial agent for Staphylococcus aureus that does not exhibit antibacterial activity against beneficial Staphylococcus epidermidis, but exhibits antibacterial activity against harmful Staphylococcus aureus. This selective antibacterial agent for Staphylococcus aureus includes not only crude extracts of the total fatty acids obtained from the cells of bacteria of the genus Bifidobacterium after the culture step, but also 7-cis-hexadecenoic acid isolated and purified from crude extracts of the total fatty acids.

In addition, the method for producing a food/beverage product for improving atopic dermatitis of the present invention comprises a step of culturing bacteria of the genus Bifidobacterium capable of producing a monounsaturated fatty acid having 16 carbon atoms, and the monounsaturated fatty acid having 16 carbon atoms is 7-cis-hexadecenoic acid. Thus, since 7-cis-hexadecenoic acid, which does not exhibit antibacterial activity against beneficial Staphylococcus epidermidis, but exhibits antibacterial activity against Staphylococcus aureus, one of the factors contributing to atopic dermatitis, is produced by bacteria of the genus Bifidobacterium, this allows to obtain a 7-cis-hexadecenoic acid-containing material that is easily used in foods and beverages. Food/beverage products containing this 7-cis-hexadecenoic acid-containing material can be utilized as a food/beverage product for improving atopic dermatitis.

In addition, the method for producing a cosmetic product for improving atopic dermatitis of the present invention comprises a step of culturing bacteria of the genus Bifidobacterium capable of producing a monounsaturated fatty acid having 16 carbon atoms, and the monounsaturated fatty acid having 16 carbon atoms is 7-cis-hexadecenoic acid. Thus, since 7-cis-hexadecenoic acid, which does not exhibit antibacterial activity against beneficial Staphylococcus epidermidis, but exhibits antibacterial activity against Staphylococcus aureus, one of the factors contributing to atopic dermatitis, is produced by bacteria of the genus Bifidobacterium, this allows to obtain a 7-cis-hexadecenoic acid-containing material that is easily used in cosmetic products. Cosmetic products containing this 7-cis-hexadecenoic acid-containing material can be utilized as a cosmetic product for improving atopic dermatitis.

Furthermore, the novel bacterium of the genus Bifidobacterium of the present invention is the Bifidobacterium sp. strain AD2 (NITE BP-03576). Cultivation of this novel Bifidobacterium sp. strain AD2 allows to highly efficiently produce monounsaturated fatty acids having 16 carbon atoms, particularly 7-cis-hexadecenoic acid.

### Advantageous Effects of Invention

The present invention can provide a method for producing a monounsaturated fatty acid having 16 carbon atoms and having the following excellent effects, as well as a method for producing a selective antibacterial agent for Staphylococcus aureus and a food/beverage product or cosmetic product for improving atopic dermatitis.
(1) Cultivation of bifidobacteria allows to produce monounsaturated fatty acids having 16 carbon atoms, particularly 7-cis-hexadecenoic acid.
(2) For culture conditions, such as medium composition, incubation temperature, and gas aeration, using suitable conditions can improve the amount of monounsaturated fatty acids having 16 carbon atoms produced.
(3) Being produced by bifidobacteria, which are used in the production of fermented food products, probiotics, and the like, it is highly safe and the products are easily used in the fields of food/beverage products and cosmetic products.

### BRIEF DESCRIPTION OF THE DRAWINGS

FIG. 1 is a graph showing the localization of 7-cis-hexadecenoic acid produced by bifidobacteria in the bacterial cells in Example 2; the vertical axis represents the amount of 7-cis-hexadecenoic acid (7-cis-C16:1) in 1 L of culture solution;

FIG. 2 is a graph showing the antibacterial activity of fatty acid extracts from bifidobacteria against Staphylococcus aureus and Staphylococcus epidermidis in Example 3;

FIG. 3(a) and FIG. 3(b) are graphs showing the amount (mg/L) of 7-cis-hexadecenoic acid (7-cis-C16:1) in each culture solution when bifidobacteria were cultured in MRS liquid medium, CSL liquid medium, and TOS liquid medium in Example 4, and the percentage (%) of 7-cis-hexadecenoic acid in the total fatty acids; FIG. 3(a) is the graph for the cultivation of Bifidobacterium sp. strain JCM7042, and FIG. 3(b) is the graph for the cultivation of Bifidobacterium boum strain JCM1211;

FIG. 4(a) and FIG. 4(b) are graphs showing the amount (mg/L) of 7-cis-hexadecenoic acid (7-cis-C16:1) in each culture solution when bifidobacteria were cultured in MRS liquid medium and MRS+L-Cys liquid medium in Example 6, and the percentage (%) of 7-cis-hexadecenoic acid in the total fatty acids; FIG. 4(a) is the graph for the cultivation of Bifidobacterium sp. strain JCM7042, and FIG. 4(b) is the graph for the cultivation of Bifidobacterium boum strain JCM1211;

FIG. 5 is a graph showing the amount (mg/L) of 7-cis-hexadecenoic acid (7-cis-C16:1) in each culture solution when a scale-up culture of bifidobacteria was performed in TOS liquid medium, MRS liquid medium, and MRS+L-Cys liquid medium by carbon dioxide or nitrogen gas aerated culture in Example 7, and the incubation time (h);

FIG. 6 is a graph showing the amount (mg/L) of 7-cis-hexadecenoic acid (7-cis-C16:1) in each culture solution when bifidobacteria were cultured in a liquid medium in which the amount of L-cysteine added to MRS liquid medium was varied in Example 8;

FIG. 7 is a graph showing the amount (mg/L) of 7-cis-hexadecenoic acid (7-cis-C16:1) in each culture solution when varying the incubation temperature of bifidobacteria in Example 9;

FIG. 8 is a graph showing the amount (mg/L) of 7-cis-hexadecenoic acid (7-cis-C16:1) in the culture solution when the incubation temperature of bifidobacteria was set to 34° C. and a scale-up culture was performed by carbon dioxide aerated culture in Example 9, and the incubation time (h); and

FIG. 9(a) and FIG. 9(b) are graphs showing the amount (mg/L) of 7-cis-hexadecenoic acid (7-cis-C16:1) in each culture solution, and FIG. 9(b) is a graph showing the amount of 7-cis-hexadecenoic acid accumulated per dry cell weight (mg/gDCW), for the wild strain (strain JCM7042) and mutant strains (strains AD1 and AD2) of bifidobacteria in Example 10.

### DETAILED DESCRIPTION OF THE PREFERRED EMBODIMENTS

The following is a detailed description of the method for producing monounsaturated fatty acids having 16 carbon atoms and the method for producing food products, beverage products and cosmetic products for improving atopic dermatitis according to one embodiment of the present invention. The method for producing monounsaturated fatty acids having 16 carbon atoms according to the present embodiment comprises a step of culturing bacteria of the genus Bifidobacterium capable of producing monounsaturated fatty acids having 16 carbon atoms in a liquid medium.

(Bacteria of the Genus Bifidobacterium)

First, the bacteria of the genus Bifidobacterium used in the present embodiment will be described. The bacteria of the genus Bifidobacterium according to the present embodiment are bacteria of the genus Bifidobacterium capable of producing monounsaturated fatty acids having 16 carbon atoms. Specifically, suitable examples include Bifidobacterium adolescentis, Bifidobacterium thermophilum, Bifidobacterium boum, Bifidobacterium ruminantium, Bifidobacterium gallinarum, Bifidobacterium dentium, Bifidobacterium breve, Bifidobacterium sp. strain JCM7042, and the mutant strains thereof. Of these, from the viewpoint of having excellent productivity of monounsaturated fatty acids having 16 carbon atoms, Bifidobacterium adolescentis, Bifidobacterium boum, Bifidobacterium sp. strain JCM7042 and the mutant strains thereof are more preferably used.

The mutant strain is not limited as long as it is capable of producing monounsaturated fatty acids having 16 carbon atoms, but as shown in the Example 10 described below, in the present invention, the Bifidobacterium sp. mutant strains AD1 and AD2 having the JCM7042 strain as the parent strain have been obtained as mutant strains having excellent productivity of monounsaturated fatty acids having 16 carbon atoms. These exhibit better productivity of monounsaturated fatty acids having 16 carbon atoms than the parent strain JCM7042. Of these, the strain AD2, i.e., Bifidobacterium sp. strain AD2, which exhibits particularly good productivity, has been deposited internationally with the Patent Microorganisms Depositary as follows.
(1) Accession number: NITE BP-03576
(2) Date of the original deposit: Dec. 22, 2021
(3) Depositary organization: Patent Microorganisms Depositary Center, National Institute of Technology and Evaluation (#122, 2-5-8 Kazusakamatari, Kisarazu-shi, Chiba 2920818)

(Monounsaturated Fatty Acid Having 16 Carbon Atoms)

In the present embodiment, the monounsaturated fatty acid having 16 carbon atoms is a monounsaturated fatty acid having 16 carbon atoms that exhibits selective antibacterial activity, i.e., exhibits antibacterial activity against harmful Staphylococcus aureus but not against beneficial Staphylococcus epidermidis. Specific examples include at least one fatty acid selected from the group consisting of sapienic acid (6-cis-C16:1), palmitoleic acid (9-cis-C16:1) and 7-cis-hexadecenoic acid (7-cis-C16:1). Of these, 7-cis-hexadecenoic acid (7-cis-C16:1), which was found to be produced by bifidobacteria and to have improved productivity in the Examples described below, is particularly preferred. 7-cis-hexadecenoic acid (7-cis-C16:1) is a fatty acid having the selective antibacterial activity described above and can be used to treat, improve or prevent dermatitis such as atopic dermatitis while maintaining healthy microflora in the skin.

(Liquid Medium)

In the present embodiment, any liquid medium can be used as the liquid medium used to culture bacteria of the genus Bifidobacterium as long as it is a liquid medium capable of culturing and growing so-called bifidobacteria. Specifically, examples include, but are not limited to, MRS medium, TOS medium, TOS propionate medium, CSL medium, BL medium, and GAM bouillon. An example of the composition of MRS liquid medium is, per liter of liquid medium, 10 g of proteose peptone, 10 g of beef extract, 5 g of yeast extract, 20 g of glucose, 1 g of polysorbate 80, 2 g of diammonium hydrogen citrate, 5 g of sodium acetate, 0.1 g of magnesium sulfate (7-hydrate), 0.05 g of manganese sulfate, and 2 g of dipotassium hydrogen phosphate, but the amount of each component may vary by ±20%, some components may be substituted with components having similar functions, and other components may be further added. An example of the composition of TOS liquid medium is, per liter of liquid medium, 10 g of tryptone, 1 g of yeast extract, 3 g of potassium dihydrogen phosphate, 4.8 g of dipotassium hydrogen phosphate, 3 g of ammonium sulfate, 0.2 g of magnesium sulfate (7-hydrate), 0.5 g of L-cysteine hydrochloride (1-hydrate), and 10 g of galactooligosaccharide, but the amount of each component may vary by ±20%, some components may be substituted with components having similar functions, and other components may be further added. An example of the composition of TOS propionate liquid medium is, per liter of liquid medium, 10 g of peptone, 1 g of yeast extract, 3 g of potassium dihydrogen phosphate, 4.8 g of dipotassium hydrogen phosphate, 3 g of ammonium sulfate, 0.2 g of magnesium sulfate (7-hydrate), 0.5 g of L-cysteine hydrochloride (1-hydrate), 15 g of sodium propionate, and 10 g of galactooligosaccharide, but the amount of each component may vary by ±20%, some components may be substituted with components having similar functions, and other components may be further added. An example of the composition of CSL liquid medium is, per liter of liquid medium, 55 g of corn steep liquor, 10 g of glucose, 1 mL of polysorbate 80, 1 g of dipotassium hydrogen phosphate, and 1 g of potassium dihydrogen phosphate, but the amount of each component may vary by ±20%, some components may be substituted with components having similar functions, and other components may be further added. An example of the composition of BL liquid medium is, per liter of liquid medium, 2.4 g of meat extract, 10 g of proteose peptone, 5 g of peptone, 3 g of soy peptone, 5 g of yeast extract, 3.2 g of liver extract, 10 g of glucose, 0.5 g of soluble starch, 1 g of dipotassium hydrogen phosphate, 1 g of potassium dihydrogen phosphate, 0.2 g of magnesium sulfate (7-hydrate), 0.01 g of ferrous sulfate (7-hydrate), 0.01 g of sodium chloride, 0.007 g of manganese sulfate, 0.2 g of defoaming agent (silicon), 1 g of polysorbate 80, and 0.5 g of L-cysteine hydrochloride (1-hydrate), but the amount of each component may vary by ±20%, some components may be substituted with components having similar functions, and other components may be further added. Furthermore, an example of the composition of GAM bouillon is, per liter of liquid medium, 10 g of peptone, 3 g of soy peptone, 10 g of proteose peptone, 13.5 g of serum digestive powder, 5 g of yeast extract, 2.2 g of meat extract, 1.2 g of liver extract, 3 g of glucose, 2.5 g of potassium dihydrogen phosphate, 3 g of sodium chloride, 5 g of soluble starch, 0.3 g of L-cysteine hydrochloride (1-hydrate), and 0.3 g of sodium thioglycolate, but the amount of each component may vary by ±20%, some components may be substituted with components having similar functions, and other components may be further added.

Of the liquid media described above, the liquid medium used for culture is preferably MRS liquid medium, from the viewpoint of improving the productivity of monounsaturated fatty acids having 16 carbon atoms, particularly 7-cis-hexadecenoic acid (7-cis-C16:1). Cultivation in MRS liquid medium can increase the concentration of 7-cis-hexadecenoic acid in the culture solution 2- to 5-fold compared to CSL liquid medium or TOS liquid medium. From the viewpoint of being able to further improve the productivity of 7-cis-hexadecenoic acid, it is particularly preferable to use MRS liquid medium to which L-cysteine or a salt thereof has been added. Cultivation in MRS liquid medium with added L-cysteine can increase the concentration of 7-cis-hexadecenoic acid in the culture solution 2-fold compared to cultivation in MRS liquid medium without L-cysteine. The amount of L-cysteine or salt thereof to be added to the MRS liquid medium is, per liter of liquid medium, preferably in the range of 0.001 to 1.0 g/L, more preferably in the range of 0.01 to 0.7 g/L, and particularly preferably in the range of 0.1 to 0.6 g/L from the viewpoint of the productivity of 7-cis-hexadecenoic acid in the culture solution.

(Culture Conditions)

In the present embodiment, when culturing bifidobacteria, it is preferable to culture under microaerobic or anaerobic conditions in which bifidobacteria easily grow, and specifically, it is preferable to perform liquid stationary culture or aerated culture. For aerated culture, it is particularly preferable to culture by aerating with carbon dioxide from the viewpoint of improving the productivity of monounsaturated fatty acids having 16 carbon atoms, particularly 7-cis-hexadecenoic acid (7-cis-C16:1). Cultivation by aerating with carbon dioxide can significantly improve the rate and amount of production of 7-cis-hexadecenoic acid. Of these, using MRS liquid medium with added L-cysteine as the liquid medium for culturing by aerating with carbon dioxide further improves the productivity of 7-cis-hexadecenoic acid, thus allowing to efficiently produce 7-cis-hexadecenoic acid in scale-up culture.

As the incubation temperature of bifidobacteria in the present embodiment, it is preferable to use temperature conditions suitable for the growth of bifidobacteria, but from the viewpoint of improving the productivity of monounsaturated fatty acids having 16 carbon atoms, particularly the productivity of 7-cis-hexadecenoic acid (7-cis-C16:1), the incubation temperature is preferably in the range of 30° C. to 37° C., more preferably in the range of 32° C. to 35° C., and still more preferably 34° C., as shown in the Examples described below.

The incubation time of the bifidobacteria in the present embodiment can be set as appropriate, and the bifidobacteria can be cultured until the desired concentration of monounsaturated fatty acids having 16 carbon atoms is reached. As an example, it is preferably about 12 hours to 5 days, and more preferably about 24 hours to 72 hours.

(Use of Culture Solution)

The monounsaturated fatty acids having 16 carbon atoms, such as 7-cis-hexadecenoic acid (7-cis-C16:1), produced by the bifidobacteria, are accumulated as polar lipids in the cells of the bifidobacteria. Therefore, when using the culture solution obtained by the culture step, it is possible to use the culture solution itself, but it is also possible to use the bacterial cells by recovering them from the culture solution by centrifugation and the like, depending on the purpose. Various additional treatments such as washing, drying such as freeze-drying, L-drying, or spray-drying, and heating can be applied to the recovered bacterial cells and culture solution as long as the effect of the present invention is not lost. The recovered bacterial cells can be used as live bacteria or as dead bacteria, and a mixture of live and dead bacteria may be used. Furthermore, it is naturally possible to extract the total fatty acids including the monounsaturated fatty acids having 16 carbon atoms such as 7-cis-hexadecenoic acid accumulated in the bacterial cells from the recovered bacteria, or to further isolate and purify the monounsaturated fatty acids having 16 carbon atoms such as 7-cis-hexadecenoic acid from the extracted total fatty acids and use them.

(Method for Producing Selective Antibacterial Agent for Staphylococcus aureus)

The method for producing the selective antibacterial agent for Staphylococcus aureus according to the present embodiment has a step of culturing bacteria of the genus Bifidobacterium capable of producing 7-cis-hexadecenoic acid in a liquid medium. The bacteria of the genus Bifidobacterium capable of producing 7-cis-hexadecenoic acid, the liquid medium used for the culture, the culture conditions, and the mode of use of the culture solution are the same as described above, and the effects thereof are also the same. This selective antibacterial agent for Staphylococcus aureus includes not only crude extracts of the total fatty acids obtained from the cells of bacteria of the genus Bifidobacterium after the culture step, but also 7-cis-hexadecenoic acid isolated and purified from crude extracts of the total fatty acids. Furthermore, specific examples of the method for producing a selective antibacterial agent for Staphylococcus aureus include a step of recovering the cells of the bacteria of the genus Bifidobacterium after the culture step and an extraction step to obtain a crude extract of the total fatty acids from the bacterial cells after the recovery step, but the method is not limited as long as a substance having selective antibacterial activity against Staphylococcus aureus is obtained. The total fatty acids are preferably extracted as free fatty acids from the total lipids of the bacterial cells. In addition, it is also possible to provide a purification step to isolate and purify 7-cis-hexadecenoic acid from the crude extract of the total fatty acids after the extraction step. This allows to obtain the selective antibacterial agent for Staphylococcus aureus, which does not exhibit antibacterial activity against beneficial Staphylococcus epidermidis, but exhibits antibacterial activity against Staphylococcus aureus, as a material derived from bifidobacteria.

(Method for Producing Food/Beverage Products and Cosmetic Products for Improving Atopic Dermatitis)

The method for producing a food/beverage product for improving atopic dermatitis or cosmetic product for improving atopic dermatitis according to the present embodiment has a step of culturing bacteria of the genus Bifidobacterium capable of producing 7-cis-hexadecenoic acid in a liquid medium. The bacteria of the genus Bifidobacterium capable of producing 7-cis-hexadecenoic acid, the liquid medium used for the culture, the culture conditions, and the mode of use of the culture solution are the same as described above, and the effects thereof are also the same. The 7-cis-hexadecenoic acid thus produced by bifidobacteria is not a chemical compound but a product of fermentation by bifidobacteria. Furthermore, since bifidobacteria are bacteria that have long been used in fermented foods such as yogurt and as probiotics to maintain human health, and are highly safe, 7-cis-hexadecenoic acid can be produced as a material that can be easily used in food/beverage products such as supplements and beverages, and cosmetic products such as emulsions and creams.

The culture solution containing a culture produced by bifidobacteria, the recovered bacterial cells or processed products thereof, or 7-cis-hexadecenoic acid extracted from the recovered bacterial cells can be used in food/beverage products of all forms such as in supplement forms such as tablets, capsules, powders, granules, and gels; in beverage products such as fermented milk, lactic acid bacteria beverages, soft drinks, and sports drinks; in dairy products such as yogurt and ice cream; confectioneries such as candies, gums, and chocolate; in bread, porridge, cereals, noodles, jellies, soups, and seasonings, and can be used for the improvement, treatment, or prevention of atopic dermatitis. It is also possible to combine these food/beverage products with other functional ingredients, other microorganisms, and nutrients such as sugars, vitamins, minerals, amino acids or proteins.

In addition, the culture solutions containing cultures produced by bifidobacteria, recovered bacterial cells or processed products thereof, or 7-cis-hexadecenoic acid extracted from recovered bacterial cells can be applied to various cosmetic dosage forms by conventional methods as a dosage form normally applied to the skin, and for example, can be used in liquid formulations such as lotions, emulsions, gels, powders, creams, patches, facial masks, cleansers, soaps, hair care products, or makeup products, which can be used for the improvement, treatment or prevention of atopic dermatitis. Various ingredients normally contained in cosmetic products can be added, as long as they do not impair the effects of the present invention, and examples thereof include moisturizing agents, emollients, plant extracts, vegetable oils and fats, pH adjusters, surfactants, thickeners, vitamins, amino acids, preservatives, fragrances, and pigments. The cosmetic product in the present specification includes not only cosmetic products but also quasi-drugs.

Hereinafter, the present invention will further be described in detail by Examples, but the present invention is not limited by these Examples in any way.

### EXAMPLES

### Example 1

1. Study of Bifidobacteria Capable of Producing Monounsaturated Fatty Acids Having 16 Carbon Atoms

In the present Example, microorganisms that produce monounsaturated fatty acids having 16 carbon atoms were searched for. In particular, microorganisms used in fermented food products that are highly safe and have a long history of use were selected and tested, so that they can be used as raw materials for food/beverage products and external skin preparations.

Specifically, the tests were performed as follows. MRS liquid medium with the composition shown in Table 1 below was dispensed into 12 mL screw-cap test tubes, inoculated with various preserved strains, and allowed to stand in the sealed screw-cap test tubes to perform liquid stationary culture. The incubation temperature was set to 37° C. and the culture was performed until reaching a turbidity of OD660=1 to obtain a pre-culture solution. Next, as the main culture, 12 mL of MRS liquid medium was dispensed into a screw-cap test tube, then 120 μL of the pre-culture solution was added, and the mixture was allowed to stand in the sealed screw-cap test tube to perform liquid stationary culture. The incubation temperature was set to 37° C., and the incubation period for the main culture was set to two days.

TABLE 1

| MRS Liquid Medium Components | Content | |
|---|---|---|
| Proteose Peptone | 10 | g |
| Beef Extract | 10 | g |
| Yeast Extract | 5 | g |
| Glucose | 20 | g |
| Polysorbate 80 | 1 | g |
| (Tween (registered trademark) 80) | | |
| Diammonium Hydrogen Citrate | 2 | g |
| Sodium Acetate | 5 | g |
| Magnesium Sulfate (7-hydrate) | 0.1 | g |
| Manganese Sulfate (n-hydrate) | 0.05 | g |
| Dipotassium Hydrogen Phosphate | 2 | g |

| | |
|---|---|
| Distilled Water | up to 1 L |

The culture solution of the main culture was centrifuged at 3260×g for 10 minutes to recover the bacteria, which were dried in an oven at 95° C. for 2 hours. The dry cell weight was calculated by subtracting the tare weight from the test tube weight after drying the bacteria. Tricosanoic acid (C23:0) was used as the internal standard and dissolved in dichloromethane to a concentration of 0.2 mg/mL. To the dried bacteria, 1 mL of dichloromethane containing the internal standard and 2 mL of 10% hydrochloric acid-methanol were added, the test tube was sealed, and incubated at 55° C. for 2 hours. This allowed to simultaneously perform fatty acid extraction from the bacteria and methyl esterification of the extracted fatty acids. After adding 1 mL of pure water and 3 mL of n-hexane and mixing vigorously, the mixture was centrifuged at 3,260×g for 10 minutes, and the upper hexane layer was transferred to a new test tube. After removing hexane by a centrifugal evaporator, the obtained fatty acid methyl ester fraction was dissolved in 100 μL of chloroform and subjected to gas chromatography (GC) analysis under the following conditions. Each fatty acid component was quantified relative to the content of the internal standard and the area value of each fatty acid peak.
Column: GC capillary column for fatty acid separation (model number: TC-70, product of GL Sciences Inc.)
Column size: inner diameter 0.25 mm×length 60 m, film thickness 0.25 pam
Column temperature: 160° C.
Heating rate: 2° C./min to 230° C.
Hold-up time: 10 minutes at 230° C.
Vaporization chamber temperature: 250° C.
Detector temperature: 250° C.
Injection volume: 1 μL

As a result, bacteria of the genus Bifidobacterium (bifidobacteria), which are yogurt-producing bacteria and are well known as probiotics, were found to produce monounsaturated fatty acids having 16 carbon atoms. The results are shown in Table 2 below. Total fatty acids (mg/L) indicates the total fatty acid amount in the culture solution, and 7-cis-C16:1 (%) indicates the percentage of 7-cis-hexadecenoic acid in the total fatty acids. The structure of the monounsaturated fatty acid having 16 carbon atoms produced by these bifidobacteria was determined and found to be 7-cis-hexadecenoic acid (7-cis-C16:1). Of the bifidobacteria, especially Bifidobacterium sp. strain JCM7042, Bifidobacterium adolescentis and Bifidobacterium boum were found to accumulate nearly 2% of the total fatty acids as 7-cis-C16:1.

TABLE 2

| | Total | | |
|---|---|---|---|
| | Fatty | 7-cis- | Dry Cell |
| | Acids | C16:1 | Weight |
| Strain | (mg/L) | (%) | (mg) |
| Bifidobacterium bifidum | 24.7 | 0.0 | 16.8 |
| Bifidobacterium sp. JCM 7042 | 106.6 | 2.3 | 36.6 |
| Bifidobacterium adolescentis JCM 1275 | 43.4 | 0.0 | 37.9 |
| Bifidobacterium adolescentis 12451 (*) | 55.8 | 1.8 | 25.8 |
| Bifidobacterium adolescentis 3-117 (*) | 54.4 | 0.5 | 27.9 |
| Bifidobacterium adolescentis 12-114 (*) | 39.4 | 0.9 | 21.8 |
| Bifidobacterium adolescentis 12-111 (*) | 106.0 | 2.3 | 35.7 |
| Bifidobacterium adolescentis 4-2 (*) | 57.4 | 0.0 | 27.2 |
| Bifidobacterium adolescentis 4-16 (*) | 19.6 | 0.0 | 9.2 |
| Bifidobacterium adolescentis 4-58 (*) | 39.0 | 0.6 | 24.0 |
| Bifidobacterium adolescentis 9-124 (*) | 33.4 | 0.3 | 20.8 |
| Bifidobacterium animalis | 44.7 | 0.0 | 21.9 |
| subspp. animalis JCM 1190 | | | |
| Bifidobacterium animalis | 35.2 | 0.6 | 25.3 |
| subspp. lactis JCM 10602 | | | |
| Bifidobacterium boum JCM 1211 | 77.5 | 2.7 | 31.5 |
| Bifidobacterium breve | 67.8 | 0.5 | 22.3 |
| Bifidobacterium catenulatum | 36.6 | 0.0 | 16.7 |
| Bifidobacterium dentium JCM 1192 | 44.4 | 0.6 | 21.6 |
| Bifidobacterium gallinarum JCM 16291 | 20.2 | 0.7 | 11.8 |
| Bifidobacterium indicum JCM 1302 | 23.4 | 0.0 | 9.9 |
| Bifidobacterium longum 105A wild | 65.4 | 0.0 | 25.8 |
| Bifidobacterium longum infantis | 38.6 | 0.0 | 20.7 |
| ATCC 15697 | | | |
| Bifidobacterium longum infantis | 56.0 | 0.0 | 40.1 |
| JCM 1222 | | | |
| Bifidobacterium longum NCC 2705 | 93.5 | 0.0 | 35.4 |
| Bifidobacterium pseudocatenulatum | 40.9 | 0.0 | 48.2 |
| Bifidobacterium ruminantium JCM 8222 | 74.9 | 0.7 | 30.6 |
| Bifidobacterium thermophilum JCM 1207 | 68.1 | 0.8 | 24.0 |

(*): Strains owned by the Laboratory of Environmental Microbial Engineering, Gifu University

### Example 2

2. Study of Localization of Monounsaturated Fatty Acid Having 16 Carbon Atoms Produced by Bifidobacteria

In the present Example, Bifidobacterium sp. strain JCM7042 was used to analyze in which lipid form 7-cis-hexadecenoic acid is accumulated in the cells of bifidobacteria producing 7-cis-hexadecenoic acid.

Specifically, the test was performed as follows. 12 mL of TOS liquid medium with the composition shown in Table 3 below was dispensed into a screw-cap test tube, inoculated with Bifidobacterium sp. strain JCM7042 (hereinafter referred to as “strain JCM7042”), and allowed to stand in the sealed screw-cap test tube to perform liquid stationary culture. The incubation temperature was set to 37° C. and the culture was performed until reaching a turbidity of OD660=1 to obtain a pre-culture solution. Next, as the main culture, 10 mL of the pre-culture solution was added to 1 L of the TOS liquid medium, and liquid stationary culture was performed with the test tube tightly sealed. The incubation temperature was set to 37° C. and the incubation period for the main culture was set to three days.

TABLE 3

| TOS Liquid Medium Components | Content | |
|---|---|---|
| Tryptone | 10 | g |
| Yeast Extract | 1 | g |
| Potassium Dihydrogen | 3 | g |
| Phosphate | | |
| Dipotassium Hydrogen | 4.8 | g |
| Phosphate | | |
| Ammonium Sulfate | 3 | g |
| Magnesium Sulfate (7-hydrate) | 0.2 | g |
| Galactooligosaccharide | 10 | g |
| L-Cysteine Hydrochloride | 0.5 | g |
| (1-hydrate) | | |

| | |
|---|---|
| Distilled Water | up to 1 L |

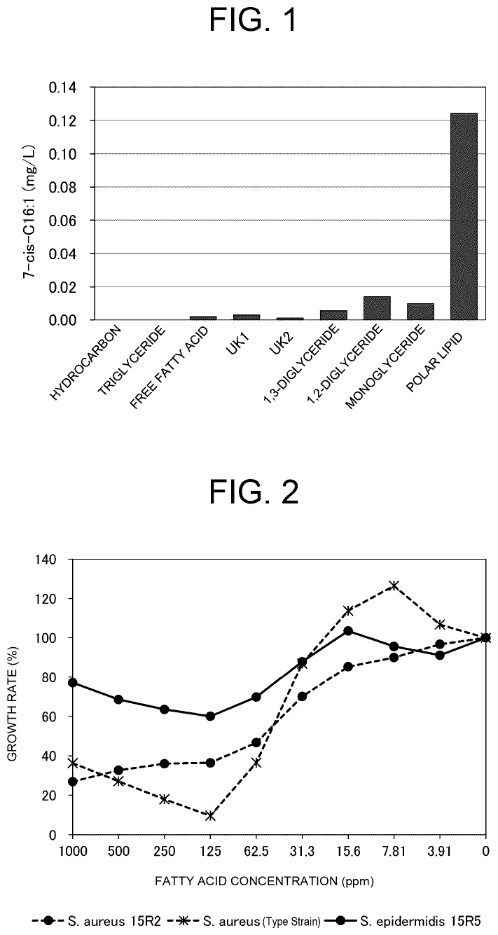

One liter of the culture solution of the main culture was centrifuged and a large amount of bacteria was recovered. Extraction of total lipids from the wet bacterial cells was performed by the Bligh-Dyer method, and water/chloroform/methanol (final ratio: 2/2.5/2.5, v/v/v) was used for the extraction. The extracted total lipids were developed with a developing solvent of hexane:diethyl ether:acetic acid=80:20:1 on thin layer chromatography, which was then sprayed with an 80% acetone solution containing 0.01% (w/v) primulin, and the lipid spots were detected by ultraviolet light at 365 nm. In this way, the lipids in the bacteria were extracted and fractionated, and the fatty acid composition and amount of each lipid species were analyzed. Many fatty acids were present as constituent fatty acids of polar lipids (mainly phospholipids), and 7-cis-hexadecenoic acid (7-cis-C16:1) was also found to be similarly contained in polar lipids, as shown in the graph of FIG. 1. Note that UK1 and UK2 in the graph of FIG. 1 indicate unknown substances.

### Example 3

3. Study of Antibacterial Activity of Fatty Acids Produced by Bifidobacteria

In the present Example, total fatty acids were extracted from the cultured cells of bifidobacteria (Bifidobacterium sp. strain JCM7042), for which effective production of 7-cis-hexadecenoic acid was confirmed in Examples 1 and 2, and the antibacterial activity of the crude extract was investigated.

Specifically, the test was performed as follows. Bifidobacterium sp. strain JCM7042 (strain JCM7042) was used as the bifidobacterium. As in Example 2, pre-culture was performed using the TOS liquid medium shown in Table 3. The incubation temperature was set to 37° C. and the culture was performed until reaching a turbidity of OD660=1.0 to obtain a pre-culture solution. Next, as the main culture, 0.14 mL of the pre-culture solution was added to 14 mL of the TOS liquid medium, and liquid stationary culture was performed with the test tube tightly sealed at 37° C. After cultivation until reaching a turbidity of OD660=1.0, the entire volume was transferred to a 15-mL tube. The bacteria were recovered by centrifuging at 4000×g for 15 minutes at 4° C. The bacteria were washed once with PBS and then suspended in 100 μL of PBS. To this was added 50 μL of 5N HCL and 400 μL of acetonitrile, and the mixture was vortexed for 1 minute, then incubated for 1 hour at 100° C., and cooled to room temperature. Next, 100 μL of methanol, 800 μL of t-butyl methyl ether, and 400 μL of ultrapure water were added and the mixture was vortexed for 1 minute. Centrifugation was performed at 300×g for 5 minutes, and the upper layer was recovered in a new 15 mL tube. To the recovered upper layer, 800 μL of ultrapure water was added, the mixture was vortexed for 1 minute, then centrifuged at 300×g for 5 minutes, and the upper layer was recovered in a new 15 mL tube. The upper layer was recovered in a dried 1.5 mL tube and dried in a centrifugal evaporator. There, the 1.5 mL tube was weighed and the amount of fatty acid extract recovered was weighed. In this way, the total fatty acids from the cells of the bifidobacteria were obtained as a crude extract of free fatty acids separated from the whole lipids.

Staphylococcus aureus and Staphylococcus epidermidis were used as targets for measuring antibacterial activity. As S. aureus, S. aureus strain JCM20624 (type strain) and S. aureus strain 15R2 were used, and as S. epidermidis, S. epidermidis strain 15R5 was used. S. aureus strain 15R2 and S. epidermidis strain 15R5 are strains isolated from the same patient with severe atopic dermatitis (strains owned by the Laboratory of Environmental Microbial Engineering, Gifu University).

Two 96-well round-bottom plates were used, the first for dilution of the fatty acid extract of bifidobacteria and the second for culture. First, the first plate was used to perform a serial dilution of the fatty acid extract. 11 μL of DMSO and 99 μL of NB medium were added to rows 3 to 10 and 12. 22 μL of 20,000 ppm fatty acid extract dissolved in DMSO and 198 μL of NB medium were added to the second row. The second row was mixed by pipetting, from which 110 μL was added to the third row. The third row was mixed by pipetting, from which 110 μL was added to the fourth row. This operation was repeated, and 110 μL of the 10th row was placed in the 11th row. The 12th row was not touched. Next, the second plate was used to prepare a bacterial solution. The pre-culture solution of the above strains cultured to OD₆₆₀=1.0 was diluted 1000 times, and the concentration of the bacterial solution was adjusted to 1.0×10⁵ CFU/mL. 100 μL of this bacterial solution was added to each of the wells of rows 2 to 10 and 12. The 11th row was used as a negative control, and was filled with 100 μL of NB medium only. 100 μL of the dilution of the fatty acid extract prepared in the first 96-well plate was added to each corresponding well of the second 96-well plate, and mixed by pipetting. In this way, the fatty acid extract in the medium was set to 10 levels of concentrations: 1000 ppm, 500 ppm, 250 ppm, 125 ppm, 63 ppm, 31 ppm, 16 ppm, 8 ppm, 4 ppm, and 0 ppm. The second plate was covered with a gas-exchangeable plate seal (Breathe-Easy, product of Diversified Biotech) and a stationary culture was performed at 37° C. After culture, the turbidity at OD₆₆₀ in each well was measured and the ratio of turbidity in each well when the turbidity at a fatty acid extract concentration of 0 ppm is 100% was determined as the growth rate (%).

FIG. 2 shows the antibacterial activity of the fatty acid extracts obtained from the cultured cells of Bifidobacterium sp. strain JCM7042. According to the results, when 60 ppm or more of this fatty acid extract are present, the growth rate of Staphylococcus aureus (S. aureus) is 50% or less, but Staphylococcus epidermidis (S. epidermidis) retained a growth rate of 60% or more over a concentration of 0 to 1000 ppm of the fatty acid extract. This indicates that this fatty acid extract containing 7-cis-hexadecenoic acid exhibits selective antibacterial activity against Staphylococcus aureus (S. aureus), the bacterium causing skin inflammation, even in this crude state. The percentage of 7-cis-hexadecenoic acid in the crude extract of fatty acids obtained in the present Example is about 2 to 4%, and therefore, the concentration of 7-cis-hexadecenoic acid at the concentration at which a decrease in growth rate by the fatty acid extract of the present Example was observed (about 60 ppm) was calculated to be about 1.2 to 2.4 μg/mL. The MIC of 7-cis-hexadecenoic acid itself against Staphylococcus aureus is about 3 μg/mL.

### Example 4

4. Improvement of Productivity of 7-Cis-Hexadecenoic Acid by Bifidobacteria (1)

The bifidobacteria producing 7-cis-hexadecenoic acid were cultured using different types of media and the relationship to the productivity of 7-cis-hexadecenoic acid was studied.

As the bifidobacteria, Bifidobacterium sp. strain JCM7042 (strain JCM7042) and Bifidobacterium boum strain JCM1211 (strain boum1211) were used and cultured in the same manner as in Example 1, except that the MRS liquid medium in Example 1 was replaced by the TOS liquid medium shown in Table 3 or the CSL liquid medium with the composition shown in Table 4 below. Using the same methods and conditions as in Example 1, the 7-cis-hexadecenoic acid in each culture solution was quantified by gas chromatography (GC) analysis.

TABLE 4

| CSL Liquid Medium Components | Content | |
|---|---|---|
| Corn Steep Liquor | 55 | g |
| Glucose | 10 | g |
| Polysorbate 80 | 1 | mL |
| (Tween (registered trademark) 80) | | |
| Potassium Dihydrogen Phosphate | 1 | g |
| Dipotassium Hydrogen Phosphate | 1 | g |

| | |
|---|---|
| Distilled Water | up to 1 L |

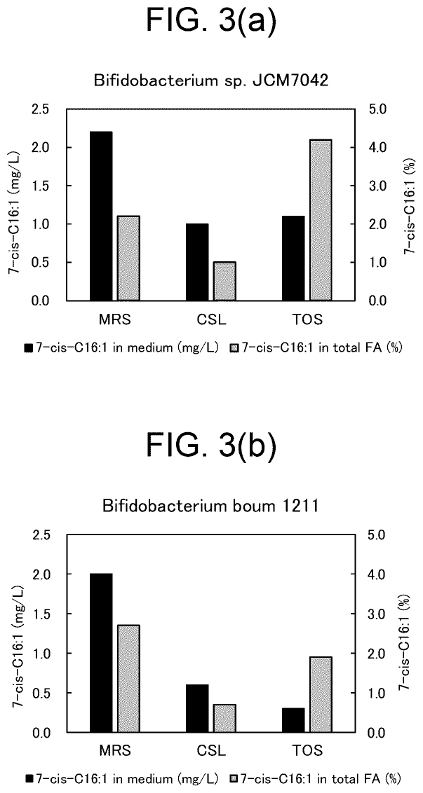

FIG. 3(a) shows the results for strain JCM7042 and FIG. 3(b) shows the results for strain boum1211. 7-cis-C16:1 (mg/L) indicates the amount of 7-cis-hexadecenoic acid in the culture solution, and 7-cis-C16:1 (%) indicates the percentage of 7-cis-hexadecenoic acid in the total fatty acids. The data for MRS liquid medium are the same as those obtained in Example 1.

The results indicate that using MRS liquid medium as the medium promoted the production of 7-cis-hexadecenoic acid by bifidobacteria, resulting in a concentration of 7-cis-hexadecenoic acid in the culture solution of 2 mg/L or more. On the other hand, it was found that, when TOS liquid medium was used, the concentration of 7-cis-hexadecenoic acid in the culture solution was lower than in MRS liquid medium, but the percentage of 7-cis-hexadecenoic acid in the total fatty acids was improved.

### Example 5

5. Improvement of Productivity of 7-Cis-Hexadecenoic Acid by Bifidobacteria (2)

The effects on the growth of bifidobacteria and the production of 7-cis-hexadecenoic acid (7-cis-C16:1) were investigated by adding or replacing components in the MRS medium and components thought to be involved in fatty acid production, to the TOS liquid medium of the composition shown in Table 3.

Specifically, the test was performed as follows. A liquid medium was prepared by adding each of the additive components shown in Table 5 below to the TOS liquid medium with the composition shown in Table 3. For the liquid medium in which the additive component was “glucose,” glucose was added while omitting galactooligosaccharide for the purpose of replacing galactooligosaccharide in the composition of the TOS liquid medium. In addition, for the liquid medium in which the additive components were “peptone” and “casamino acids,” peptone and casamino acids were added while omitting tryptone for the purpose of replacing tryptone in the composition of the TOS liquid medium. Furthermore, for the liquid medium in which the additive component was “tryptone,” indicated by the symbol * in Table 5, since the TOS liquid medium originally contains “tryptone,” a liquid medium with a composition omitting tryptone from the composition of the TOS liquid medium was prepared for comparison and a control test was conducted. Similarly, for the liquid medium in which the additive component was “L-cysteine hydrochloride,” indicated by the symbol * in Table 5, since the TOS liquid medium originally contains “L-cysteine hydrochloride,” a liquid medium with a composition omitting L-cysteine hydrochloride from the composition of the TOS liquid medium was prepared for comparison and a control test was conducted.

As the bifidobacteria, Bifidobacterium sp. strain JCM7042 (strain JCM7042) was used and cultured in the same manner as in Example 1, except that the MRS liquid medium in Example 1 was replaced by the various liquid media prepared as described above. Using the same methods and conditions as in Example 1, the 7-cis-hexadecenoic acid (7-cis-C16:1) in the various culture solutions was quantified by gas chromatography (GC) analysis. The growth of bifidobacteria was evaluated as “excellent” when the cell weight increased, “good” when the cell weight was maintained, “fair” when the cell weight decreased, and “poor” when the cell weight decreased significantly, compared to the control without additive components. Moreover, the production of 7-cis-hexadecenoic acid (7-cis-C16:1) was evaluated as “excellent” when the produced amount of 7-cis-hexadecenoic acid increased, “good” when the produced amount was maintained, “fair” when the produced amount decreased, and “poor” when the produced amount decreased significantly. The additive components “tryptone” and “L-cysteine hydrochloride,” were evaluated by comparing with the results of the control test in a liquid medium without these components. The results are shown in Table 5 below.

TABLE 5

| | Amount added | | | Bifido- | 7-cis- |
|---|---|---|---|---|---|
| Component | (per liter of | | Substituted | bacteria | C16:1 |
| Added | medium) | | Component | Growth | Production |
| None (Control) | — | | — | Good | Good |
| Glucose | 10 | g | Galacto- | Good | Good |
| | | | oligo- | | |
| | | | saccharide | | |
| Tryptone | 10 | g | * | Good | Good |
| Peptone | 10 | g | Tryptone | Fair | Fair |
| Casamino | 10 | g | Tryptone | Good | Good |
| Acids | | | | | |
| L-Cysteine | 0.5 | g | * | Excellent | Excellent |
| Hydrochloride | | | | | |
| (1-hydrate) | | | | | |
| Oxaloacetic | 10 | g | — | Good | Poor |
| Acid | | | | | |
| Calcium | 1 | g | — | Excellent | Fair |
| Carbonate | | | | | |
| Potassium | 1 | g | — | Poor | Poor |
| Carbonate | | | | | |
| Sodium | 1 | g | — | Poor | Poor |
| Carbonate | | | | | |
| Potassium | 1 | g | — | Poor | Fair |
| Hydrogen | | | | | |
| Carbonate | | | | | |
| Sodium Hydrogen | 1 | g | — | Poor | Poor |
| Carbonate | | | | | |
| Polysorbate 80 | 0.1 | g | — | Good | Poor |
| (Tween | | | | | |
| (registered | | | | | |
| trademark) 80) | | | | | |
| Triton X-100 | 0.1 | g | — | Poor | Poor |
| Tergitol NP40 | 0.1 | g | — | Good | Good |
| Anisidine | 0.1 | g | — | Good | Good |
| Saponin | 0.1 | g | — | Good | Good |
| Lauric Acid | 0.1 | g | — | Poor | Poor |
| (free form) | | | | | |
| Lauric Acid | 0.1 | g | — | Good | Good |
| Methyl Ester | | | | | |
| Myristic Acid | 0.1 | g | — | Poor | Poor |
| (free form) | | | | | |
| Myristic Acid | 0.1 | g | — | Poor | Poor |
| Methyl Ester | | | | | |
| Palmitic Acid | 0.1 | g | — | Poor | Poor |
| (free form) | | | | | |
| Palmitic Acid | 0.1 | g | — | Poor | Poor |
| Methyl Ester | | | | | |
| Oleic Acid | 0.1 | g | — | Fair | Poor |
| (free form) | | | | | |
| Oleic Acid | 0.1 | g | — | Excellent | Good |
| Methyl Ester | | | | | |

These results clearly indicate that L-cysteine is an essential amino acid in the culture of bifidobacteria and has the effect of improving the growth of bifidobacteria, but also has the effect of improving the productivity of 7-cis-hexadecenoic acid as well. Note that, for the liquid medium to which calcium carbonate or oleic acid methyl ester was added, it was shown that either there was no effect on the amount of 7-cis-hexadecenoic acid produced, or the amount produced was decreased, even though the growth of the bifidobacteria was improved.

### Example 6

6. Improvement of Productivity of 7-Cis-Hexadecenoic Acid by Bifidobacteria (3)

The possibility of further improving the productivity of 7-cis-hexadecenoic acid was studied by combining the liquid media and additive components which showed a high productivity of 7-cis-hexadecenoic acid in Examples 4 and 5.

Specifically, the test was performed as follows. Bifidobacterium sp. strain JCM7042 (strain JCM7042) and Bifidobacterium boum strain JCM1211 (strain boum1211) were used as the bifidobacteria. The MRS liquid medium shown in Table 1 and MRS+L-Cys liquid medium in which L-cysteine hydrochloride monohydrate was added at 0.5 g/L to the MRS liquid medium shown in Table 1 were prepared, and a pre-culture and main culture were performed in these two liquid media in the same manner as in Example 1. Using the same methods and conditions as in Example 1, the 7-cis-hexadecenoic acid in each main culture solution was quantified by gas chromatography (GC) analysis.

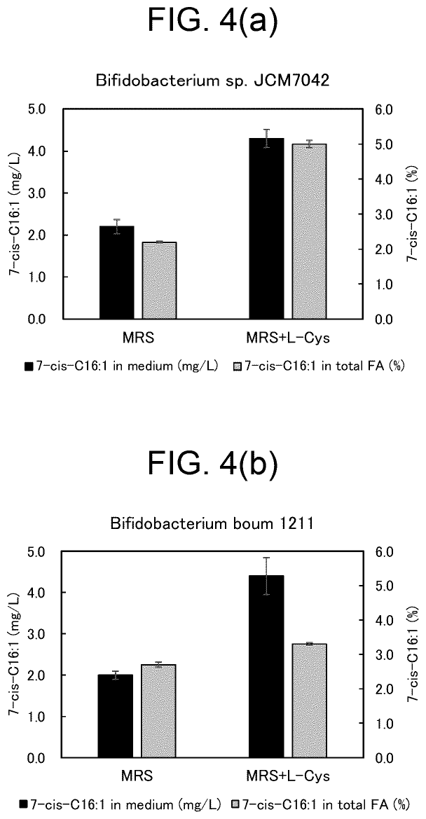

FIG. 4(a) shows the results for strain JCM7042 and FIG. 4(b) shows the results for strain boum1211. 7-cis-C16:1 (mg/L) indicates the amount of 7-cis-hexadecenoic acid in the culture solution, and 7-cis-C16:1 (%) indicates the percentage of 7-cis-hexadecenoic acid in the total fatty acids. The data for MRS liquid medium are the same as those obtained in Example 1.

The results indicate that using a liquid medium in which L-cysteine is further added to MRS liquid medium as the medium promoted the production of 7-cis-hexadecenoic acid by bifidobacteria, resulting in a concentration of 7-cis-hexadecenoic acid in the culture solution of more than 4 mg/L. In addition, it was found that the percentage of 7-cis-hexadecenoic acid in the total fatty acids was also improved.

### Example 7

7. Improvement of Productivity of 7-Cis-Hexadecenoic Acid by Bifidobacteria (4)

The culture conditions for increasing the productivity of 7-cis-hexadecenoic acid when performing a scale-up culture of bifidobacteria were investigated.

Specifically, the test was performed as follows. Bifidobacterium sp. strain JCM7042 (strain JCM7042) was used as the bifidobacterium. The MRS liquid medium shown in Table 1, MRS+L-Cys liquid medium in which L-cysteine hydrochloride monohydrate was added at 0.5 g/L to the MRS liquid medium shown in Table 1, and the TOS liquid medium shown in Table 3 were each prepared, and a pre-culture was performed using each liquid medium. The incubation temperature was set to 37° C. and the culture was performed until reaching a turbidity of OD₆₆₀=1 to obtain a pre-culture solution. Next, as a main culture, 30 mL of the pre-culture solution was added to a 3.5 L medium bottle containing 3 L of the same liquid medium as the pre-culture solution. The culture was performed at 37° C. while aerating the medium bottle with pure carbon dioxide or pure nitrogen gas and stirring at 700 rpm with a stirrer. Each culture solution was collected with a syringe at a predetermined sampling time, and 5 mL of the culture solution was centrifuged to collect the bacteria. Using the same methods and conditions as in Example 1, the 7-cis-hexadecenoic acid in each culture solution was quantified by gas chromatography (GC) analysis.

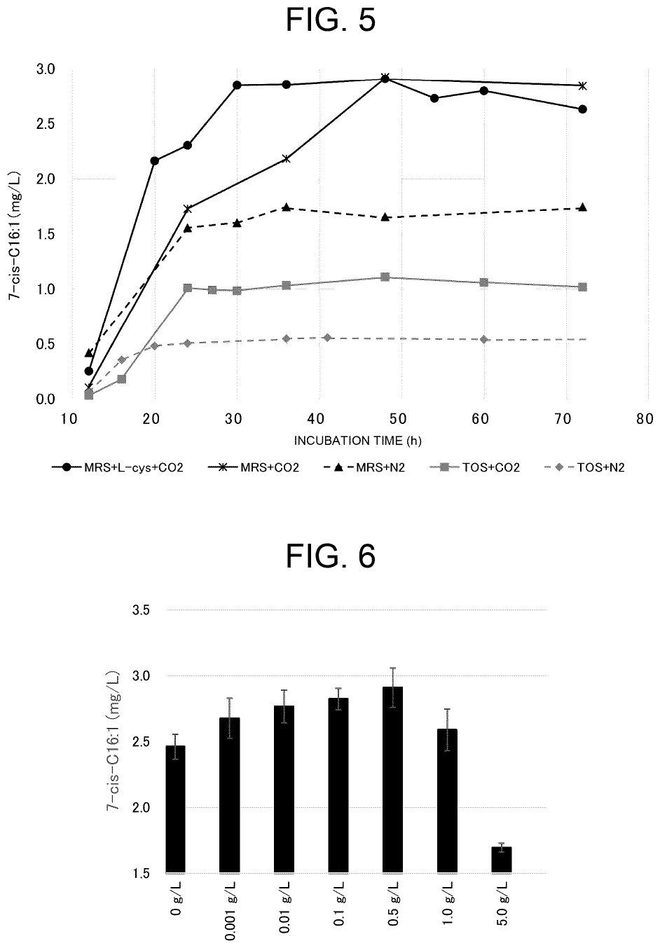

The results are shown in FIG. 5. In the graph of FIG. 5, the solid line and black circle markers indicate the results of the culture in MRS+L-Cys liquid medium aerated with carbon dioxide, the solid line and star markers indicate the results of the culture in MRS liquid medium aerated with carbon dioxide, the dashed line and triangle markers indicate the results of the culture in MRS liquid medium aerated with nitrogen gas, the solid line and square markers indicate the results of the culture in TOS liquid medium aerated with carbon dioxide, and the dashed line and diamond markers indicate the results of the culture in TOS liquid medium aerated with nitrogen gas. The vertical axis indicates the amount of 7-cis-hexadecenoic acid in the culture solution, and the horizontal axis indicates the time (h) that the main culture was performed.

The results of this scale-up culture test clearly indicate that the maximum accumulation of 7-cis-hexadecenoic acid was approximately doubled by aerating with carbon dioxide compared to the test section aerated with nitrogen gas, indicating that the amount of 7-cis-hexadecenoic acid produced was greatly improved. Furthermore, it was found that the addition of L-cysteine to the MRS liquid medium increased the rate of production of 7-cis-hexadecenoic acid and approximately halved the time to reach maximum accumulation compared to the MRS medium without added L-cysteine.

### Example 8

8. Study of L-Cysteine Concentration in Liquid Medium

MRS media with various amounts of L-cysteine added were prepared to investigate their effect on the production of 7-cis-hexadecenoic acid by bifidobacteria. Specifically, the test was performed as follows. Bifidobacterium sp. strain JCM7042 (strain JCM7042) was used as the Bifidobacterium. L-cysteine hydrochloride monohydrate was added at 0.001 g/L, 0.01 g/L, 0.1 g/L, 0.5 g/L, 1.0 g/L, and 5.0 g/L to the MRS liquid medium with the composition shown in Table 1 to prepare L-cysteine-containing MRS liquid media. These MRS liquid media with L-cysteine and the control MRS liquid medium (without added L-cysteine) were cultured in the same manner as in Example 1. Using the same methods and conditions as in Example 1, the 7-cis-hexadecenoic acid in each culture solution was quantified by gas chromatography (GC) analysis.

The results are shown in FIG. 6. These results show that a concentration of L-cysteine hydrochloride in the liquid medium as high as 5.0 g/L greatly inhibited the production of 7-cis-hexadecenoic acid. Therefore, it was estimated that the concentration of L-cysteine hydrochloride in the liquid medium is preferably in the range of 0.001 to 1.0 g/L, and more preferably in the range of 0.01 to 0.7 g/L.

### Example 9

9. Study of Incubation Temperature

The main culture was performed at varying incubation temperatures to investigate their effect on the production of 7-cis-hexadecenoic acid by bifidobacteria. Specifically, the test was performed as follows. Bifidobacterium sp. strain JCM7042 (strain JCM7042) was used as the bifidobacterium. Using the MRS liquid medium with the composition shown in Table 1, pre-culture was performed at 37° C. in the same manner as in Example 1. Next, when performing the main culture, the incubation temperatures were set in the range of 28° C. to 37° C., and the main culture was performed in the same manner as in Example 1, except that the culture was performed at each incubation temperature. Using the same methods and conditions as in Example 1, the 7-cis-hexadecenoic acid in each culture solution was quantified by gas chromatography (GC) analysis.

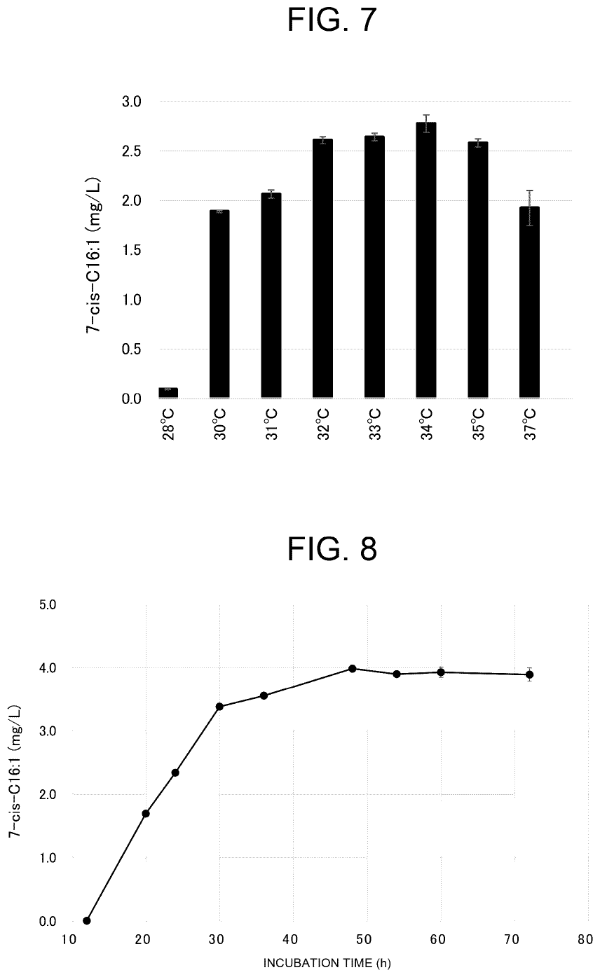

The results are shown in FIG. 7. These results show that the preferred incubation temperature for improving the productivity of 7-cis-hexadecenoic acid is 30° C. to 37° C. Of these, particularly the range of 32° C. to 35° C. showed excellent productivity of 7-cis-hexadecenoic acid, with the concentration of 7-cis-hexadecenoic acid in the culture solution reaching over 2.5 mg/L.

Then, a test was conducted to establish whether the productivity of 7-cis-hexadecenoic acid could be further improved by performing a scale-up culture aerated with carbon dioxide using the same procedures and methods as in Example 7, except for changing the incubation temperature of the main culture to 34° C. in the scale-up culture performed in Example 7 described above. Bifidobacterium sp. strain JCM7042 (strain JCM7042) was used as the bifidobacterium, and MRS+L-Cys liquid medium in which L-cysteine hydrochloride monohydrate was added at 0.5 g/L to the MRS liquid medium shown in Table 1 was used as the liquid medium.

The results are shown in FIG. 8. The vertical axis indicates the amount of 7-cis-hexadecenoic acid in the culture solution, and the horizontal axis indicates the time (h) that the main culture was performed. The results indicate that cultivation at 34° C. further improved the productivity of 7-cis-hexadecenoic acid, with the concentration of 7-cis-hexadecenoic acid in the culture solution reaching 4 mg/L.

### Example 10

10. Creation of a Strain with Enhanced 7-Cis-Hexadecenoic Acid Production by Mutagenesis

In the present Example, it was attempted to obtain a strain with enhanced 7-cis-hexadecenoic acid production by the mutagenesis method. Specifically, the test was performed as follows. Bifidobacterium sp. strain JCM7042 (strain JCM7042) was used as the parent bifidobacterium strain. 12 mL of TOS liquid medium with the composition shown in Table 3 was dispensed into a screw-cap test tube, inoculated with the strain JCM7042, and allowed to stand in the sealed screw-cap test tube to perform liquid stationary culture. The incubation temperature was set to 37° C. and the culture was performed until reaching a turbidity of OD₆₆₀=1. To 1 mL of this culture solution, 10 μL of ethyl methanesulfonate was added (final concentration: 1% (v/v)), and the mixture was mixed, then incubated at 37° C. for 1 hour to perform a mutagenesis treatment.

Nile Red was selected as the indicator in the present test. Nile Red is incorporated into the lipid fraction of the bacterial cells, giving it a red color. Therefore, it is possible to obtain a mutant strain with improved ability to produce fatty acids by picking up the colonies that turned redder and redder faster on agar medium. 100 μL of the bacterial solution after the mutagenesis treatment was applied on TOS agar medium coated with this Nile Red on the surface, and anaerobic culture was performed at 37° C. using a deoxygenating and carbon dioxide generating agent (AnaeroPack (registered trademark) Kenki, product of Mitsubishi Gas Chemical Company, Inc.). In the present test, the grown colonies with a strong red coloration were picked up in priority. For each picked up strain, liquid culture was performed in the same manner as in Example 1, except that it was cultured in TOS liquid medium. Using the same methods and conditions as in Example 1, the components of each fatty acid in each culture solution were quantified by gas chromatography (GC) analysis.

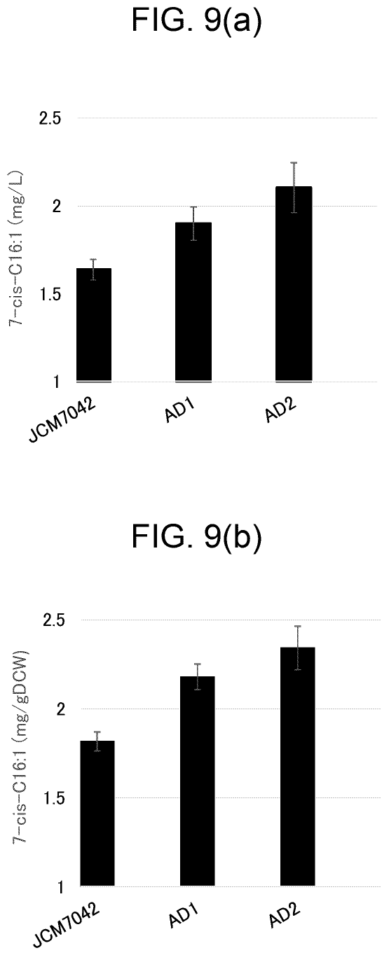

As a result, through primary and secondary screening, it was possible to obtain two mutant strains that exhibited higher 7-cis-hexadecenoic acid productivity than the wild strain, strain JCM7042. FIG. 9(a) shows the amount (mg/L) of 7-cis-hexadecenoic acid in the culture solution of the wild strain (strain JCM7042) and the mutant strains, strains AD1 and AD2, cultured in TOS liquid medium. FIG. 9(b) shows the amount of 7-cis-hexadecenoic acid accumulated per dry cell weight (mg/gDCW). Of these, the AD2 strain which had the highest amount of 7-cis-hexadecenoic acid produced was deposited with the Patent Microorganisms Depositary Center as NITE BP-03576.

The present invention is not limited to the above embodiments or Examples, and also includes within its technical scope various design modifications within the scope not deviating from the gist of the invention described in the claims.

### INDUSTRIAL APPLICABILITY

The present invention provides a monounsaturated fatty acid having 16 carbon atoms that exhibits selective antibacterial activity, i.e., exhibits antibacterial activity against harmful Staphylococcus aureus but not against beneficial Staphylococcus epidermidis, and a food product or cosmetic product containing the same, and is useful in a wide range of industries in the fields of food products, cosmetic products, quasi-drugs, pharmaceuticals, and the like.

### Accession Number

Accession number: NITE BP-03576, Bifidobacterium sp. strain AD2; date of the original deposit: Dec. 22, 2021; depositary organization: Patent Microorganisms Depositary Center, National Institute of Technology and Evaluation (#122, 2-5-8 Kazusakamatari, Kisarazu-shi, Chiba 2920818)

What is claimed is:

1. A method for producing a monounsaturated fatty acid having 16 carbon atoms, comprising a step of culturing bacteria of the genus Bifidobacterium capable of producing a monounsaturated fatty acid having 16 carbon atoms in a liquid medium.

2. The method for producing a monounsaturated fatty acid having 16 carbon atoms according to claim 1, wherein the bacterium of the genus Bifidobacterium is Bifidobacterium adolescentis, Bifidobacterium boum, Bifidobacterium sp. strain JCM7042 or a mutant strain thereof.

3. The method for producing a monounsaturated fatty acid having 16 carbon atoms according to claim 2, wherein the mutant strain is Bifidobacterium sp. strain AD2 (NITE BP-03576).

4. The method for producing a monounsaturated fatty acid having 16 carbon atoms according to claim 2, wherein the monounsaturated fatty acid having 16 carbon atoms is 7-cis-hexadecenoic acid.

5. The method for producing a monounsaturated fatty acid having 16 carbon atoms according to claim 2, wherein the liquid medium is a liquid medium in which 0.001 to 1 g/L of L-cysteine or a salt thereof is further added to MRS medium.

6. The method for producing a monounsaturated fatty acid having 16 carbon atoms according to claim 2, wherein the culture is performed by aerating the liquid medium with carbon dioxide.

7. The method for producing a monounsaturated fatty acid having 16 carbon atoms according to claim 2, wherein the culture is performed under temperature conditions of 32 to 35° C.

8. A method for producing a selective antibacterial agent for Staphylococcus aureus, comprising a step of culturing bacteria of the genus Bifidobacterium capable of producing a monounsaturated fatty acid having 16 carbon atoms in a liquid medium, and
wherein the monounsaturated fatty acid having 16 carbon atoms is 7-cis-hexadecenoic acid.

9. A method for producing a food/beverage product or a cosmetic product for improving atopic dermatitis, comprising a step of culturing bacteria of the genus Bifidobacterium capable of producing a monounsaturated fatty acid having 16 carbon atoms in a liquid medium, and wherein the monounsaturated fatty acid having 16 carbon atoms is 7-cis-hexadecenoic acid.

10. Bifidobacterium sp. strain AD2 (NITE BP-03576).
